(19) United States
(12) Patent Application Publication
(10) Pub. No.: US 2005/0118641 A1
(43) Pub. Date: Jun. 2, 2005

(54) Design Technique for Use in Engineering of Deamidation Rates of Peptides, Proteins, Hormones, and Peptide-Like, Protein-Like and Hormone-Like Molecules

(75) Inventor: Noah E. Robinson, Cave Junction, OR (US)

Correspondence Address: Noah E. Robinson, 2251 Dick George Rd., Cave Junction, OR, 97523, US

(73) Assignee: Noah E. Robinson, Cave Junction, OR (US)

(21) Appl. No.: 10/707,263

(22) Filed: Dec. 2, 2003

(51) Int. Cl. G01N 33/53; G06F 19/00; G01N 33/48; G01N 33/50

(52) U.S. Cl. 435/7.1; 702/19

(57) ABSTRACT

A method that quantitatively explains the primary sequence dependence of deamidation has been invented on the basis of a simple steric and catalytic model. Application of this method to the known deamidation rates of peptides has produced a table of coefficients that permits prediction of primary sequence deamidation rates and provides that ability to engineer amides with specific rates. This work permits a better understanding of deamidation, provides a prediction procedure for protein engineering, and will allow improved computation of peptide and protein primary, secondary, and tertiary structure deamidation rates. Not only can this method be used for engineering amides, but it should also work for isomerization and racemization of carboxylic acids.

### BACKGROUND OF INVENTION

The deamidation of peptides and proteins as well as molecules related to peptides and proteins is a well known phenomenon. In this reaction Asn or Gln residues are gradually changed into Asp and Glu residues respectively as well as their isomers and charged products. The rate of this reaction is dependent on the primary sequence, three-dimensional structure, pH, temperature, buffer type, ionic strength and other solution properties. The half-time varies from less than 1 day to more than a century. The reaction introduces a negative charge into the molecule. In addition, the isomerization products β-Asp and β-Glu as well D-isomerized forms and chain cleavage also accompany the reaction.

The stability of Asn and Gln in pharmaceutical and other types of commercial preparations is a major field of study. Efforts have been made to discover formulation conditions that will control the rate of deamidation of amides in these preparations.

### SUMMARY OF INVENTION

The inventions described here allow the engineering of molecules with specific amide structures that will deamidate at a specified rate. This procedure can be used to design stable and unstable forms for pharmaceutical, industrial, and other products. This can be used to increase the shelf-life of such products through minor modifications, prevent or at least slow down the gradual formation of impurities in preparations with these modifications, and may make possible as a result of minor modifications the use of products that would otherwise be too unstable for practical purposes. The engineering of products with unstable amides that are programmed to deamidate at specific rates may also be a valuable application of this procedure.

This invention is based on newly discovered rules that govern the deamidation rates of amides. These rules can be derived using a newly discovered method. Many of them have been derived and more can be derived using this method. These rules involve the effects of steric and catalytic factors in the nearby proximity of amides. It has been shown that these rules can be quantitatively applied to determine the deamidation rates of amides to a very high accuracy. Even without the exact use of these rules, the qualitative effect on the deamidation rate of adding or subtracting various functional groups in various positions has been discovered.

This procedure makes available a wide variety of possible variations that will modify deamidation rates. Modification of the residue to the right of the amide using either natural or non-natural amino acids, as well as modification of Asn or Gln and determination of the quantitative effects on the deamidation rate are now available.

Insulin is an example of an important pharmaceutical molecule that deamidates during storage. The procedure described in this patent allows the invention of stable forms of insulin that require a minimum of modifications and will likely have the same biological activity as the native form. Two specific modifications are given as examples. Many other pharmaceutical preparations, such as interferon and tumor necrosis factor are thought to be deactivated by deamidation.

### DETAILED DESCRIPTION

General Method:

It was known before the invention of the method described here, that the sequence and structure around an amide has a large effect on the deamidation rate. What was unknown was how this effect worked or any quantitative information that would allow engineering of stable or unstable amides or amides with predetermined rates. As a result of experiments that I carried out, sufficient data to allow such a method to be invented were collected. The method described here uses this data and makes clear how more such data can be applied to further extend the method. It has been developed using Asn and Gln residues in peptides. It applies to peptides, hormones, proteins, and peptide-like, hormone-like, protein-like molecules, as well as other molecules that have unstable amides in similar surroundings. It also applies to isomerization and racemization of Asp and Glu in similar circumstances to Asn and Gln. No such system has, to my knowledge, ever been described.

This method involves two parts. The first of these is the use of experimental data to determine the effect of atoms and structures made up of atoms on the effective activation energy of the deamidation reaction. Each type of structure is assumed to increase or decrease the overall activation energy of the reaction and is assigned a coefficient depending on its location in the structure. The second part involves the use of these derived coefficients to predict primary sequence deamidation rates for use in engineering amides with particular rates or stable structures. Even without the quantitative basis, this method makes clear the required modifications necessary to achieve a particular qualitative change in a deamidation, isomerization, or racemization rate.

Two types of deamidation are obviously present. The one on which this method is based, and which is most prevalent for amides with half-times less than a few hundred days, depending on conditions and providing especially catalytic ions are not present, is most strongly effected by the structure to the right of the amide (e.g. in the sequence GlyXxx(Amide/Acid)YyyGly the identity of Yyy is the most important factor). Also present is at least one more mechanism that is usually slower and has different sequence dependence. It is possible that this dependence as well as the left hand structure dependence (Xxx in the sequence GlyXxx(Amide/Acid)YyyGly) can also be modeled with a similar system, but this has not yet been demonstrated.

Most of the structures that increase the activation energy and therefore slow the rate, apparently do so through steric hindrance of the reaction. Sometimes both a catalytic and a steric effect are present in a particular atom or group of atoms.

This model is based on the understanding that an individual molecule deamidates only as a statistical phenomenon. It does not deamidate at a particular rate. Each amide has a probability that it will deamidate over a particular time interval. The combined statistics of an ensemble of such molecules produce the deamidation rate. For example, the deamidation rate of GlyXxxAsnGlyGly is about 1 day, so each individual molecule has a 50% chance of deamidating in 1 day or in one second only a 0.00058% chance of deamidating. The deamidation of even a fast amide occurs infrequently.

Regardless of the details of the mechanism, this method assumes the amide must adopt a particular conformation in order to react. Sterically hindering groups have at least two ways of interfering. Any atom or group of atoms interfering at the reaction site can increase the energy required for the amide to move into position and raise the activation energy. Also, when such a structure can adopt more than one conformation it may interfere to a significant extent only a certain fraction of the time. Steric effects, therefore, depend on group size, the conformations available to the group, and the distribution function of occupation of these conformations. These ideas are used to understand steric effects.

Calculation of Constants:

Median values for carboxyl side residues of Asn and Gln were used in these derivations. The measurements for Asn are the most reliable and are used for these derivations. Gln calculations are likely to contain significantly more experimental error, as well as the possibility of contributions from other types of deamidation mechanisms, but are still commercially useable. An average correction for the effect thought to be the largest, hydrolysis, has been made to all rates, but only significantly affects the Gln rates and very slow Asn rates. The half-time of hydrolysis was assumed to be 8010 days, but may be faster or slower. Regardless of the derivation based on Asn, calculations of Gln based on these values are found to approximately agree with the currently available data.

The natural log of the median hydrolysis-corrected rate constants times 100 (s⁻¹) for the Asn and Gln pentapeptides as a function of carboxyl-side residue as computed from the experimental data are listed in Table 1. The first columns are (100)(ln(k)) and are directly proportional to the Arrhenius activation energy. The second columns are normalized to Gly. This leaves only the side-chain effects and also defines the effect of the Gly hydrogen side-chain or a similar atom with mostly steric effects in this β-position as zero.

TABLE 1
(100)ln(k) deamidation for the medians of
Xxx in GlyXxx(Asn/Gln)YyyGly^{#}

| | Asn^{§} | | Gln^{§} | |
|---|---|---|---|---|
| | Calculated | Normalized | Calculated | Normalized |
| Yyy | Values | to Gly | Values | to Gly |
| Gly | −1186.3 | 0 | −1831.1 | 0 |
| His | −1405.8 | 219.4 | −2181.5 | 350.4 |
| Ser | −1448.3 | 262.0 | −2165.6 | 334.4 |
| Ala | −1493.1 | 306.8 | −2178.2 | 347.1 |
| Asp | −1520.1 | 333.7 | −2393.4 | 562.3 |
| AmCys | −1528.4 | 342.1 | — | |
| Thr | −1556.9 | 370.6 | −2136.5 | 305.3 |
| Cys | −1566.2 | 379.8 | −1958.8 | 127.6 |
| Lys | −1579.4 | 393.1 | −2184.9 | 353.7 |
| Met | −1582.8 | 396.5 | −2104.7 | 273.5 |
| Glu | −1587.4 | 401.1 | −2313.2 | 482.0 |
| Arg | −1587.0 | 400.7 | −2290.7 | 459.5 |
| Phe | −1598.3 | 411.9 | −2433.6 | 602.5 |
| Tyr | −1611.5 | 425.1 | — | |
| Trp | −1630.3 | 444.0 | — | |
| Leu | −1652.6 | 466.3 | −2198.9 | 367.7 |
| Val | −1724.8 | 538.5 | −2230.9 | 399.7 |
| Ile | −1747.4 | 561.1 | −2210.2 | 379.0 |

^{§}A correction of 8.010 = half-time for hydrolysis was appled to the experimental values before normalization to Gly.
^{#}k = sec⁻¹.

Effect of Aliphatic Side-Chains:

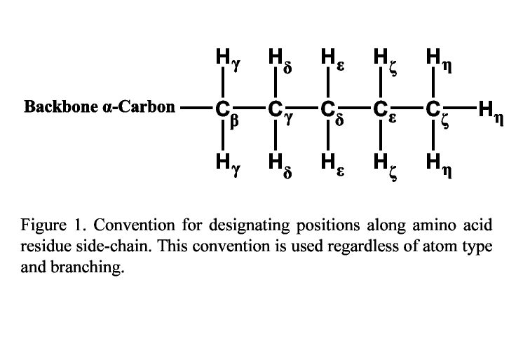

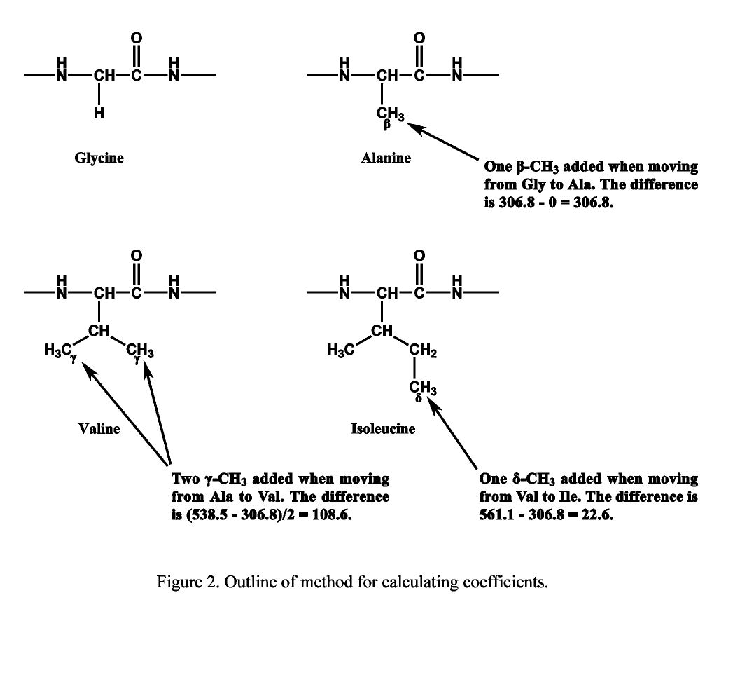

Aliphatic side-chains are assumed to have only steric effects. Any catalytic effects that might be present are masked by the steric effects. The calculations of constants for groups in each position are done by finding the difference in activation energy on structures for which a group was substituted. For the purposes of this report the α-carbon is the backbone carbon in the peptide chain with successive positions assigned Greek letters as shown in FIG. 1. FIG. 2 illustrates these calculations for three different substitutions. In some cases more than one method of calculation is possible. In all such cases the values are found to be similar and are averaged. Continuing with the method shown in FIG. 2 we can make the following calculations:
β-CH₃: (Ala Gly)=306.8 0=306.8
γ-CH₃: (Val Ala)/2=(538.5−306.8)/2=115.9
γ-CH₃: (Thr Ser)=370.6−262.0=108.6
Average γ-CH: (115.9+108.6)/2=112.3
δ-CH₃: (Ile Val)=561.1−538.5=22.6
δ-CH₃: (Leu(Ala+γ-CH₃))=466.3 306.8 112.3=23.6
Average δ-CH: (22.6+23.6)/2=23.1

Substitution increments for replacement of a hydrogen atom by a methyl group in the β, γ, and δ positions are therefore 306.8, 112.3, and 23.1. The internal consistency with two redundant examples verifies the procedure. The values become successively smaller as the methyl groups are placed farther from the reaction site as expected.

These values can also be derived from the statistical probability that the methyl groups will be in a position to interfere with deamidation. Define the effect of the β-CH₃ group as X, and substitute one of its hydrogen atoms with a γ-CH₃ group. Reference to a CPK model shows that the new methyl group will occupy a similarly interfering position for about 120° out of the 360° rotation around the α-β bond. Thus the effect of the second methyl group should be about (1/3)(X). Examination of CPK models with the addition of a δ-CH₃ group indicates that the interference will be the same as that of the γ-CH₃ except that it occurs for 180° out of the 360° of the α-β bond rotation, but only during 120° of the 360°β-γ rotation. Thus the effect of replacing a hydrogen atom with a δ-CH₃ group is (180/360−120/360)(120/360)X=(3/6−2/6)(1/3)=(1/18)X.

Since X is 306.8, the effects of the γ and δ methyl groups should be 102.3, and 17.0 respectively. In fact they are 112.3 and 23.1. Now the hydrogen atom and carbon atom effects can be separated. For example, the substitution of a γ-CH₃ group involves the removal of a γ-H and replacement by a γ-C and 3 δ-H atoms. The individual effects can be separated. The experimental results can be encapsulated in four equations:
H^{β}=0(By definition from normalizing to Gly)
C_{β}+3H_{γ}−H_{β}=306.8
C_{β}+3H_{γ}−H_{β}=112.3
C_{δ}+3H_{ε}−H_{δ}=23.1

To aid in solving these equations the carbon atom effect is postulated to be roughly a constant, “a”, times the effect of a hydrogen atom if they are in identical positions. While not a perfect approximation it is sufficient for most purposes. Thus:
C_{β}=a(H_{β})
C_{γ}=a(H_{γ})

C_{δ}=a(H_{δ})
C_{ε}=a(H_{ε})

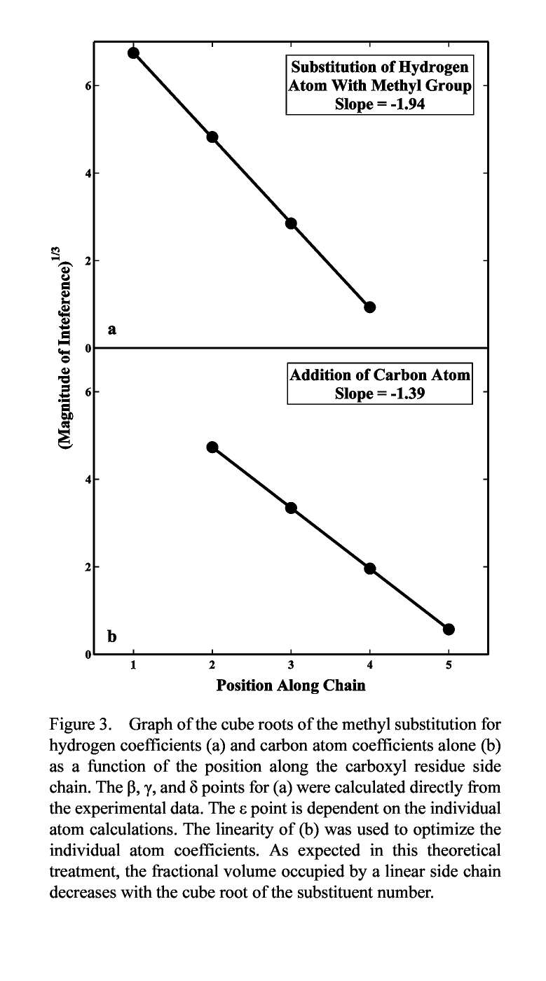

To determine the value of “a”, the cube root of the carbon atom result is plotted against the substituent number in FIG. 3b. By repetitive solving of the equations it is found that the graph becomes perfectly linear when a=1.038. Using this value for “a”:
H_{β}=0
C_{β}=0
H_{γ}=(306.8)/3=102.3
C_{γ}=a(306.8)/3=106.1
H_{δ}=(112.3+(1−a)(306.8)/3)/3=36.1
C_{δ}=a(112.3+(1−a)(306.8)/3)/3=37.5
H_{ε}=(23.1+(1−a)(112.3+(1−a)(306.8)/3)/3)/3=7.2
C_{ε}=a(23.1+(1−a)(112.3+(1−a)(306.8)/3)/3)/3=7.5

This calculation also extracts the c hydrogen and carbon atom effects even though they have not yet been measured exactly. Extrapolation of the line from the first three points in FIG. 3b also generates a value for the fourth point at ζ.

The assumption on which FIG. 3b is based is that the fractional volume occupied by an atom or group should decrease with approximately the cube of the substituent number. The unmodified experimental values derived previously from only differences in sequence rates are also of interest, but are only known in terms of removal of a hydrogen atom and substitution of a methyl group. This plot is shown in FIG. 3a and has a correlation coefficient of 0.99996. The first three values are the experimental values derived previously, and the last one is computed from the results of the calculation. The position of this fourth value optimizes at the same value of “a” where the line in FIG. 3b becomes a perfect fit.

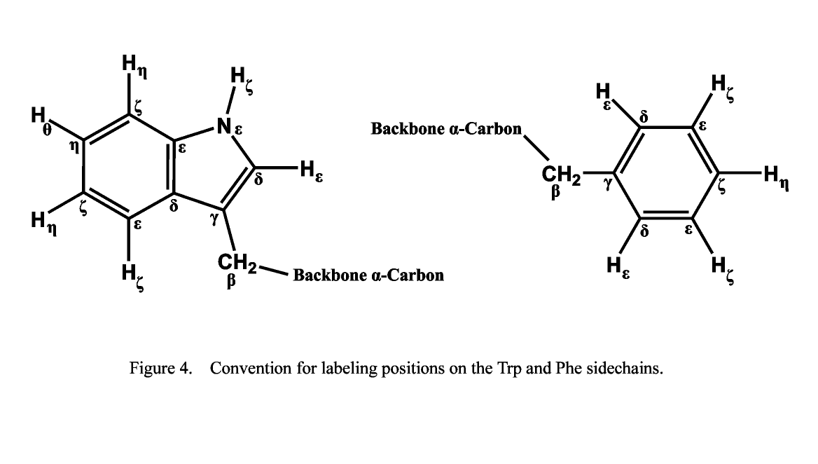

Aromatic Rings: The amino acids Phe, Tyr, and Trp contain aromatic rings. FIG. 4 shows that these rings are connected in the y position for Phe and Trp. Tyr is similar and only differs from Phe by the addition of an OH group. Using the previous results the effect of these groups can be isolated:
γC₅H₅=Phe−(C_{β}+2H_{γ})=411.9(0+2(102.3))=207.3
γC₅H₅OH=Tyr−(C_{β}+2H_{γ})=425.1(0+2(102.3))=220.5
γC₈H₆N=Trp−(C_{β}+2H_{γ})=444.0(0+2(102.3))=239.4

It also turns out that the aliphatic prediction scheme can give very accurate results for these aromatic rings without measuring them directly.

Looking at the composition of the Phe ring in FIG. 4, it is found that the γ_C and δ-C are about as free for these purposes as in a non-aromatic ring. The ε-H is not as free to move, but its direction allows it to interfere with deamidation. The remaining atoms are directed away from the reaction site. Therefore:
γ-Phenyl=C_{γ}+2C_{δ}+2H=106.1+(2)(37.5)+(2)(7.2)=195.5

If the ε carbon atoms are included, γ-Phe=210.5

Either result agrees closely with the experimental value for γ-Phe of 207.3.

For Trp the extra effect of the second ring can be added to the experimental value of 207.3 for Phe. Specifically it is necessary to determine the effect of the ζ-H atom. When averaged over the many conformations available to an aliphatic the value is very small, but this atom in Trp is held in one position that is directed toward the reaction site. FIG. 4 shows that the ζ-H atom occupies a position relative to the reaction site that is very similar to the nearby δ-C. Using the value for a δ-H atom and correcting the ε-H to a ε-C:
γ-Indole=γ-Phenyl+H_{δ}+C_{ε}−H_{ε}=207.3+36.1+7.5 7.2=243.7

Direct calculation without reference to Phe yields:
γ-Indole=C_{γ}+2C_{δ}+H_{ε}+C_{ε}+H_{δ}=106.1+(2)(37.5)+7.2+7.5+36.1=231.9

Both results are in good agreement with the experimental value of 239.4.

Tyr is identical to Phe except for the OH group in the para position on the phenyl ring. The steric hindrance should be similar to that of Phe. Tyr turns out to be slightly higher at 220.5 vs. 207.3. Why this slight modification in rate occurs is unknown, but it is exerting some type of catalytic effect, which this calculation has quantitated.

Thus the aliphatic side chains are reduced to individual atomic contributions. They can be summed to predict the deamidation rate for any sp³ hydrocarbon chain. These atomic coefficients also work well for aromatic rings. Therefore, the imide deamidation rates for any aliphatic Asn amino-side residue can be accurately computed. This ability is extremely valuable in terms of its practical quantitative prediction and engineering possibilities and in the understanding that will allow qualitative modifications to be made and understood.

Other Atoms and Charges:

Other commonly occurring atoms in the ordinary amino acid residue side chains include oxygen, nitrogen, and sulphur. Many other possible atoms exist for non-natural side-chains. In addition to steric hindrance, these atoms can have additional characteristics such as hydrogen bonding capabilities and positive and negative charges that can modify their contributions.

With only data from the naturally occurring amino acids some of these effects can be described and utilized. Further experimentation and analysis using these procedures can lead to understanding the effects of any atom, group of atoms, or similar structure both on the immediate side chain of the right hand residue and in proximity from other effects. It may be found that under certain circumstances the coefficients for particular combinations of groups cannot be directly added to determine the rate. This will entail a very minor modification of the procedures.

Of particular interest is positive charge in Arg, Lys and His. Assuming that the steric hindrance of N is similar to C we calculate:
Catalytic His=His Phe=219.4 411.9=−192.5
Catalytic Lys=Lys (C_{β}+2H_{γ}+C_{γ}+2H_{γ}+C_{γ}+2H_{ε}+C_{ε}+N_{ζ}+All others zero)=393.1[0+(2)(102.3)+106.1+(2)(36.1)+37.5+(2)(7.2)+7.5+0.2]=−49.4
Catalytic Arg=Arg(C_{β}+2H_{γ}+C_{γ}+2H_{δ}+N_{δ}+H_{ε}+C_{ε}+2N_{ζ}+All others zero)=400.7[0+(2)(102.3)+106.1+(2)(36.1)+37.5+(7.2)+7.5+(2)(0.2)]=−34.6

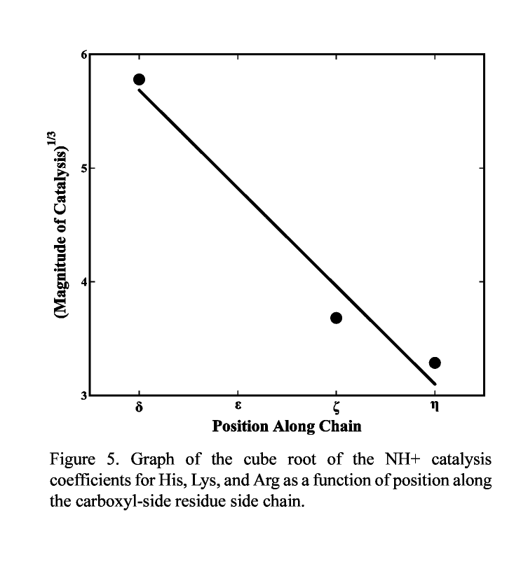

FIG. 5 illustrates these catalytic effects as a function of the distance from the reaction site. Two other points derived from rates on peptides with His one and two residues removed from the right hand side are included.

The γ-OH groups of Ser and Thr have coefficients of 55.6 (Ser is 57.4 and Thr is 53.8, this is the average). Assuming a steric effect similar to that of CH, the catalysis of this group is 55.6−142.2=−86.6.

Cys and Met contain sulfur. In the case of Cys the net effect of the SH group is 201.5. Removal of the steric effect by assuming it is similar to a CH₂ yields 201.5 178.4−36.1=−13. For AmCys (hydrogen atom replaced by acetamidomethyl group) the sulfur atom has a net effect of 84.4 (assuming no special effects from the acetamidomethyl group). Correcting for the steric effect gives 84.4−178.4=−94. For Met the net effect of S is 5.5. Correcting for its steric effect yields 5.5−52.0=−46.5.

The carboxylic acids, Asp and Glu, both have negative charges as well as hydrogen bonding capabilities. The net effect of the carboxyl is 129.2 for Asp and 15.4 for Glu. Correcting for the steric effects results in a catalytic effect of 129.2−106.1−2(37.5)=−51.9 for Asp and 15.4−37.5−2(7.5)=−37.1.

The derived increments are listed in Table 2. For convenience in protein engineering, all values listed are the net effects of the group without separation of steric and catalytic components. The catalytic effect can be isolated by subtracting the corresponding steric effects. Values listed in bold are computed exactly from experimental rates. The others are computed based on certain assumptions. The bases for the aromatics are the hydrocarbon effects shown previously to be quite accurate. The 0 values are based on both carboxylic acids and hydroxyl groups.

Gln Deamidation:

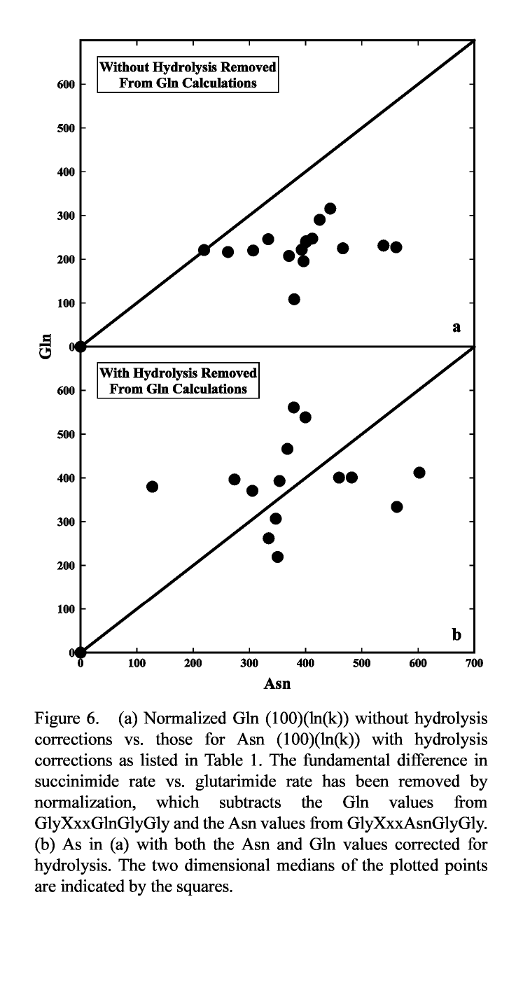

This model for Asn deamidation is also applicable to Gln deamidation, although Gln deamidation rates have not been as well characterized. While there may be important differences in the functional group effects for the longer Gln chain, the steric effects are similar as illustrated in FIG. 6. FIG. 6a, shows the Gln values without correction for hydrolysis vs. the Asn values with hydrolysis removed, as listed in Table 1. The fundamental difference between Asn and Gln rates has been removed by subtracting the Gln values from GlyXxxGlnGlyGly and the Asn values from GlyXxxAsnGlyGly.

The difference between GlyXxxGlnGlyGly and GlyXxxAsnGlyGly is 644.8. In a similar manner to the effects of steric hindrance the extra —CH₂— decreases the probability of the Gln side chain reacting. One way to look at this is by determining the extra entropy associated with the longer Gln chain. Based on the standard entropy of formation of hydrocarbons, the entropy associated with adding an extra —CH₂— is 40 J/(K mol), which corresponds to about Δ100(ln k)=480 on our scale. This is 74% of the actual experimental value.

With an average effect of hydrolysis removed, the points scatter around the theoretical 1:1 line as shown in FIG. 6b. Without this correction FIG. 6a shows the correlation to be poor. The two dimensional medians of these values are shown.

Use in Molecular Engineering:

Table 2 is a listing of the currently determined coefficients. While the addition of more values to this table would be useful, this table already allows prediction of deamidation rates based on the structure around the amide and protein engineering of many amide rates.

TABLE 2
Δ (100)ln(k) coefficients for claculating deamidation rates*.

| | β | γ | δ | ε | ζ | η |
|---|---|---|---|---|---|---|
| —H | 0 | 102.3 | 36.1 | 7.2 | 0.18 | 0 |
| —CH₃ | 306.8 | 214.5 | 59.2 | 8.1 | 0.19 | 0 |
| —CH₂ | 204.5 | 178.4 | 52.0 | 7.9 | 0.19 | 0 |
| —CH— | 102.3 | 142.2 | 44.7 | 7.7 | 0.19 | 0 |
| —C— | 0 | 106.1 | 37.5 | 7.5 | 0.19 | 0 |
| —C₅H₅ | 284.5 | 207.4 | 52.9 | 7.9 | 0 | 0 |
| —C₅H₅OH | — | 220.6 | — | — | — | — |
| —C₈H₆N (Indole) | 390.6 | 239.4 | 60.4 | 8.1 | 0 | 0 |
| —C₃H₃N₂⁺(Imidazole) | — | 14.9 | — | — | — | — |
| —S— | — | 84.4 | 5.5 | — | — | — |
| —SH | — | 201.5 | — | — | — | — |
| —O— | — | 19.5 | 11.5 | −9.6 | — | — |
| —OH | — | 55.6 | — | — | — | — |
| —CO₂⁻ | — | 129.2 | 18.2 | — | — | — |
| —NH⁺ | — | — | −136.0 | — | −49.7 | −42.1 |
| —N₃CH₅⁺(Guanidino) | — | — | — | −34.2 | — | — |
| —NH₃⁺ | — | — | — | — | −49.7 | — |

*Bold-face values based directly on experimental rates and k = sec⁻¹.

Various groups are listed in addition to individual atoms for convenience. To use this table sum up the coefficients for the carboxyl-side residue or for non-peptide like structures the groups in similar relation to the amide. Multiply bonded groups should be treated as described for Phe and Trp. The deamidation half-time is computed as follows:
Asn Peptides t_{1/2}=[(ln(2))/86400]e^{((Sum/}100)+11.863)
Gln Peptides t_{1/2}=[(ln(2))/86400]e^{((Sum/100)+18.311)}

For most Asn peptides the computed result should be within 10% or less of the experimental value in 37° C., pH 7.4, 0.15 M Tris-HCl buffer or similar conditions. Gln rates are more affected by hydrolysis and cannot be verified to as high an accuracy with the current data. For Gln peptides and long lived Asn peptides, correction for hydrolysis should be made. With a hydrolysis value of 8010 days:
t_{1/2total}=1/(1/8010+1/t_{1/2calculated})

As an example take the sequence GlyXxxAsnThrGly. To calculate the deamidation half-time from Table 2, it is necessary to consider the Thr side-chain. The structure is CH(CH₃)OH. Adding a β-CH, a γ-CH₃, and a γ-OH and computing for Asn:
^{AsnThr} succinimide t_{1/2}=[(ln(2))/86400]e^{((102.3+214.5+55.6)/100+}=47.2 days

Including hydrolysis:
AsnThr total t_{1/2}=1/((1/47.2)+(1/8010))=46.9 days

The experimental value is 46.2 days (1).

For the non-natural amino acid norleucine in the sequence AsnNle, the side chain is —CH₂CH₂CH₂CH₃. Adding the appropriate groups and computing gives:
AsnNle succinimide t_{1/2}=[(ln(2))/86400]e^{((204.5+178.4+52.0+8.1)/+11.863)}=95.6 days

Correcting for hydrolysis:
AsnNle total t_{1/2}=1/((1/95.5)+(1/8010))=94.4 days

Thus the deamidation rate of AsnNle has been determined without actually measuring it.

For molecular engineering purposes it is often easiest to work from an existing structure. Without knowing the factors that contribute to the original deamidation rate, the effects of a modification can be computed or estimated.

As an example, consider the sequence Phe-ValAsn(B3)GlnHis of insulin. This peptide deamidates at Asn(B3) with a half-time of 136 days at 37° C., pH 7.4 sodium acetate, 0.1% methyl paraben, 0.7% NaCl.

Deamidation of insulin at this position in pharmaceutical insulin preparations is a substantial problem. Many attempts have been made to modify insulin storage conditions to suppress this deamidation.

This computation method provides a procedure for slowing insulin deamidation with a minimum modification of its structure. The Gln side chain is CH₂CH₂CONH₂. Addition of two methyl groups would change this to C(CH₃)₂CH₂CONH₂.

Without reference to the primary sequence rate of AsnGln or insulin secondary and tertiary structure effects, the half-time can be computed from the current experimental rate of 136 days. Converting the 136 value to ln(k) and subtracting from the AsnGly value yields:
−1186.3−100[ln[((ln2)/136)(1/60)(1/60)(1/24)]=478.3

The modification involves removing two hydrogen atoms and replacing them by two methyl groups. The sum is:
478.3 2γ-H+2 γ-CH₃=478.3(2)(102.3)+(2)(214.5)=702.7

Substituting into the deamidation half-time calculation yields:
t_{1/2}=[(ln(2))/86400]e^{(702.7/100+11.863)}=1283 days

This modification should have a half-time increased by nearly a factor of 10 with a minimal modification in structure and involves no changes in side chain lengths or functional groups. This insulin is likely to have biological activity equivalent to the rapidly deamidating form.

### Conclusions

The methods and results described here provide a new quantitative understanding of primary sequence control of deamidation. With this quantitative understanding the effects of modifications in structure around an amide become clear, and even qualitative modifications can be undertaken with confidence and without precise calculation.

1. The invention of a technique for the design of changes in peptide and peptide-like sequences with specific deamidation rates using the following steps:
(a) The determination of a table of constants for atoms or groups or groups of atoms. This table already has been made for many functional groups and can be expanded.
(b) The summing up of constants from the table in (a) depending on type of functional group and location in residue or residue-like structure to the right of the amide.
(c) The conversion of the summed constants from (b) into a deamidation rate for the primary sequence being considered.
(d) Where other types of secondary, tertiary or quaternary structures effect the rate, the modification of the rate derived in (c) to include these parameters.

2. The invention of a method to qualitatively slow down or accelerate deamidation rates by predictable amounts through addition or subtraction of atoms or groups of atoms in the vicinity of the amide without direct reference to a table of values.

3. The use of the techniques described in claims 1 and/or 2 to design amide structures, peptide or peptide-like structures, hormone or hormone-like structures, or protein or protein-like structures with specific modifications of deamidation rates or designed deamidation rates as a part of a pharmaceutical, industrial or other type of preparation or process.

4. The use of the techniques described in claims 1 and/or 2 to modify existing chemical structures either already in use in pharmaceutical or industrial applications, currently existing for potential use in these applications or not yet invented, using the techniques in claims 1 and/or 2 to either speed up or slow down deamidation rates.

5. The use of the techniques described in claims 1 and/or 2 to stabilize molecules for pharmaceutical, industrial, or other types of preparations or processes.

6. As an example of these applications the invention of stable forms of insulin with respect to deamidation at Asn(B3) of the ValAsn(B3)Gln sequence that involve:
(a) Addition of a methyl group to the Gln(B4) side-chain to produce the side chain CH(CH3)CH2CONH2- which will have a slowed deamidation rate.
(b) Addition of 2 methyl groups to the Gln(B4) side-chain to produce the side chain C(CH3)2CH2CONH2- which should have a deamidation rate even slower than the structure described in (a).
(c) Other modifications using the techniques of claims 1 and 2 to modify the Gln(B4) side chain.
